(19) United States
(12) Patent Application Publication
(10) Pub. No.: US 2026/0296969 A1
(43) Pub. Date: Oct. 1, 2026

(54) MICROBIAL MINERALIZED MODIFIED RECYCLED AGGREGATE, AND PREPARATION METHOD AND USE THEREOF

(71) Applicant: YANTAI UNIVERSITY, Yantai (CN)

(72) Inventors: Zuowei LIU, Yantai (CN); Yuange MA, Yantai (CN); Jingzhou LU, Yantai (CN); Guang LI, Shanghai (CN)

(73) Assignee: YANTAI UNIVERSITY, Yantai (CN)

(21) Appl. No.: 19/568,103

(22) Filed: Mar. 16, 2026

(30) Foreign Application Priority Data
Mar. 28, 2025 (CN) 202510381958.8

(51) Int. Cl. C04B 20/02; C04B 103/00; C12N 1/20

(52) U.S. Cl. CPC C04B 20/02; C12N 1/20; C04B 2103/0001

(57) ABSTRACT

Provided are a microbial mineralized modified recycled aggregate, and a preparation method and use thereof. A method for preparing the microbial mineralized modified recycled aggregate includes: subjecting a culture of microorganisms having both nitrification and denitrification capabilities and a culture of ureolytic bacteria to a first mixing to obtain a mixed culture, where a volume ratio at equal concentrations of the culture of the microorganisms having both nitrification and denitrification capabilities to the culture of the ureolytic bacteria in the mixed culture is in a range of 2:1 to 1:4; and subjecting the mixed culture, a recycled aggregate and a mineralizing solution to a second mixing, and subjecting a resulting system to microbial mineralization to obtain the microbial mineralized modified recycled aggregate, where the mineralizing solution includes urea and calcium formate.

### CROSS REFERENCE TO RELATED APPLICATION

This patent application claims the benefit and priority of Chinese Patent Application No. 202510381958.8 filed with the China National Intellectual Property Administration on Mar. 28, 2025, the disclosure of which is incorporated by reference herein in its entirety as part of the present application.

### TECHNICAL FIELD

The present disclosure belongs to the technical field of building materials, and specifically relates to a microbial mineralized modified recycled aggregate, and a preparation method and use thereof.

### BACKGROUND

With the advancement of urbanization and the development of civil construction and transportation infrastructure projects, the volume of construction and demolition wastes, especially waste concrete, has been rising steadily. At present, the construction and demolition wastes are mainly disposed of in open piles or by landfilling, which, however, could cause serious environmental pollution. Conventional microbial mineralization methods are prone to producing ammonia gas, making them unsuitable for large-scale industrial modification of recycled aggregate.

Recycling the waste concrete into the recycled aggregate for use in concrete not only solves the disposal problem of the construction and demolition wastes, but also conserves natural resources. The use of the recycled aggregate shows many environmental benefits, but compared with natural aggregate, the recycled aggregate has lower apparent density, and higher water absorption and crushing value. When the natural aggregate is replaced with the recycled aggregate to produce concrete, inferior properties of the recycled aggregate result in a significant decrease in the core performance and technical indicators of recycled aggregate concrete, such as compressive strength, flexural strength and frost resistance, compared with natural aggregate concrete. Further, due to the above inherent weakness of the recycled aggregate, a replacement ratio of the recycled aggregate for the natural aggregate is usually limited to no more than 40% when preparing recycled concrete, to ensure the mechanical and durability properties of the recycled concrete, which in turn greatly restricts the utilization efficiency of the recycled aggregate. In order to improve the properties of the recycled aggregate, strengthening treatment is required. Researchers have found that applying ureolytic bacteria-induced biomineralization to the recycled aggregate could enhance its performance. However, according to “Occupational Exposure Limits for Hazardous Agents in the Workplace” (GBZ2.1-2019), a short-term exposure limit (STEL) for ammonia gas is 30 mg/m³ (about 16.7 ppm), and a permissible 8-hour time-weighted average concentration (TLV) is 20 mg/m³ (about 11 ppm). Treating 1 kg of the recycled aggregate with ureolytic bacteria leads to 75.7 ppm ammonia in a 20 L container. An amount of the ammonia gas produced in this way is too high for large-scale strengthening treatment of the recycled aggregate.

### SUMMARY

In view of this, the present disclosure provides a microbial mineralized modified recycled aggregate, and a preparation method and use thereof. Less ammonia gas is generated during preparation of the microbial mineralized modified recycled aggregate by the preparation method provided by the present disclosure, facilitating industrial application.

In order to solve the above technical problems, the present disclosure provides a method for preparing a microbial mineralized modified recycled aggregate, the method including the following steps:
subjecting a culture of microorganisms having both nitrification and denitrification capabilities and a culture of ureolytic bacteria to a first mixing to obtain a mixed culture, where a volume ratio at equal concentrations of the culture of the microorganisms having both nitrification and denitrification capabilities to the culture of the ureolytic bacteria in the mixed culture is in a range of 2:1 to 1:4; and
subjecting the mixed culture, a recycled aggregate and a mineralizing solution to a second mixing, and subjecting a resulting system to microbial mineralization to obtain the microbial mineralized modified recycled aggregate, where the mineralizing solution includes urea and calcium formate.

In some embodiments, the microorganisms having both nitrification and denitrification capabilities include at least one selected from the group consisting of Paracoccus denitrificans, and Pseudomonas. Sp; and the ureolytic bacteria include at least one selected from the group consisting of Sporosarcina pasteurii, and Bacillus megaterium de Bary.

In some embodiments, the culture of the microorganisms having both nitrification and denitrification capabilities has a concentration of 10⁷-10⁸ CFU/L (colony-forming unit per liter); and the culture of the ureolytic bacteria has a concentration of 10⁷-10⁸ CFU/L.

In some embodiments, the second mixing includes:
subjecting the mixed culture and the recycled aggregate to a third mixing, and then standing to obtain a recycled aggregate attached with the microorganisms; and
subjecting the mineralizing solution and the recycled aggregate attached with the microorganisms to a fourth mixing.

In some embodiments, a mass concentration of the urea in the mineralizing solution is in a range of 6-30 g/L, and a mass concentration of the calcium formate in the mineralizing solution is in a range of 40-45 g/L; and a ratio of a mass of the recycled aggregate to a volume of the mineralizing solution is in a range of 1 kg: 1.1-1.8 L; and a mass ratio of the mixed culture to the recycled aggregate is in a range of 0.08-0.12:1.

In some embodiments, the mineralizing solution further includes at least one selected from the group consisting of calcium chloride, and glucose;
under a condition that the calcium chloride is included in the mineralizing solution, a mass concentration of the calcium chloride in the mineralizing solution is in a range of 50-60 g/L; and
under a condition that the glucose is included in the mineralizing solution, a mass concentration of the glucose in the mineralizing solution is in a range of 0.8-1.2 g/L.

In some embodiments, after the second mixing, the method further includes: mixing the resulting system after the second mixing with a pH regulator to adjust a pH value of the resulting system to 8.5-9.5, where the pH regulator includes a sodium hydroxide solution.

In some embodiments, the microbial mineralization is conducted by standing the resulting system after the second mixing for 7-10 days at a temperature of 20-40° C.

The present disclosure further provides a microbial mineralized modified recycled aggregate prepared by the method described in the above technical solutions, where the microbial mineralized modified recycled aggregate has a water absorption of 8.58-8.82%, an apparent density of 2571-2591 kg/m³ and a crushing index of 17.5-18.1%.

The present disclosure further provides use of the microbial mineralized modified recycled aggregate described in the above technical solutions in a recycled aggregate concrete.

The present disclosure provides a method for preparing a microbial mineralized modified recycled aggregate, the method including the following steps: subjecting a culture of microorganisms having both nitrification and denitrification capabilities and a culture of ureolytic bacteria to a first mixing to obtain a mixed culture, where a volume ratio at equal concentrations of the culture of the microorganisms having both nitrification and denitrification capabilities to the culture of the ureolytic bacteria in the mixed culture is in a range of 2:1 to 1:4; and subjecting the mixed culture, a recycled aggregate and a mineralizing solution to a second mixing, and subjecting a resulting system to microbial mineralization to obtain the microbial mineralized modified recycled aggregate, where the mineralizing solution includes urea and calcium formate. In the present disclosure, the recycled aggregate is subjected to microbial mineralization under the synergistic action of the microorganisms having both nitrification and denitrification capabilities and the ureolytic bacteria at a specific ratio. During the microbial mineralization, the ureolytic bacteria decompose urea to produce ammonia gas and calcium carbonate, and the calcium carbonate attaches to a surface of the recycled aggregate to increase the density and hardness of the recycled aggregate. The microorganisms having both nitrification and denitrification capabilities convert the ammonia gas, produced by decomposition of the urea by the ureolytic bacteria, into nitrate ions and then into nitrogen gas to reduce production of the ammonia gas, and meanwhile generate calcium carbonate to attach to the surface of the recycled aggregate, further enhancing the strength of the recycled aggregate. During the microbial mineralization, a dense calcium carbonate protective layer is formed on the surface of the recycled aggregate, which not only enhances the strength of the recycled aggregate but also reduces water absorption thereof, thereby improving the performance of recycled aggregate concrete.

### BRIEF DESCRIPTION OF THE DRAWINGS

FIG. 1 shows a scanning electron microscope (SEM) image of the surface of the recycled aggregate in Example 1;

FIG. 2 shows an SEM image of the surface of the microbial mineralized modified recycled aggregate prepared in Example 3; and

FIG. 3 shows an X-ray diffraction (XRD) pattern of the microbial mineralized modified recycled aggregate prepared in Example 3.

### DETAILED DESCRIPTION OF THE EMBODIMENTS

The present disclosure provides a method for preparing a microbial mineralized modified recycled aggregate, the method including the following steps:
subjecting a culture of microorganisms having both nitrification and denitrification capabilities and a culture of ureolytic bacteria to a first mixing to obtain a mixed culture, where a volume ratio at equal concentrations of the culture of the microorganisms having both nitrification and denitrification capabilities to the culture of the ureolytic bacteria is in a range of 2:1 to 1:4; and
subjecting the mixed culture, a recycled aggregate and a mineralizing solution to a second mixing, and then subjecting a resulting system to microbial mineralization to obtain the microbial mineralized modified recycled aggregate, where the mineralizing solution includes urea and calcium formate.

In the present disclosure, a culture of microorganisms having both nitrification and denitrification capabilities and a culture of ureolytic bacteria are subjected to a first mixing to obtain a mixed culture. As a specific embodiment of the present disclosure, the microorganisms (SND bacteria) having both nitrification and denitrification capabilities include at least one selected from the group consisting of Paracoccus denitrificans, and Pseudomonas. Sp, specifically a mixture of the Paracoccus denitrificans and the Pseudomonas. Sp, the Paracoccus denitrificans, or the Pseudomonas. Sp; the Paracoccus denitrificans is Paracoccus denitrificans (Pd) (ATCC 19367), and the Pseudomonas. Sp is Pseudomonas. Sp (Psp) (ATCC 13867). As a specific embodiment of the present disclosure, the ureolytic bacteria include at least one selected from the group consisting of Sporosarcina pasteurii, and Bacillus megaterium de Bary, specifically are at least one selected from the group consisting of a mixture of the Sporosarcina pasteurii (Bacillus pasteurianus) and the Bacillus megaterium de Bary, the Sporosarcina pasteurii, and the Bacillus megaterium de Bary; the Sporosarcina pasteurii is Sporosarcina pasteurii (SP) (ATCC 11859) and the Bacillus megaterium de Bary is Bacillus megaterium de Bary (Bm) (ATCC 25300). As a specific embodiment of the present disclosure, a volume ratio at equal concentrations of the culture of the microorganisms having both nitrification and denitrification capabilities to the culture of the ureolytic bacteria in the mixed culture is in a range of 2:1 to 1:4, specifically 1:4, 1:2, 1:1 or 2:1.

As a specific embodiment of the present disclosure, a culture broth for culturing the microorganisms having both nitrification and denitrification capabilities includes a nutrient broth powder, sodium chloride, sodium nitrate and distilled water; a mass concentration of the nutrient broth powder in the culture broth for culturing the microorganisms having both nitrification and denitrification capabilities is in a range of 1-10 g/L, and further 5-8 g/L; a mass concentration of the sodium chloride in the culture broth for culturing the microorganisms having both nitrification and denitrification capabilities is in a range of 5-20 g/L, and further 10-15 g/L; and a mass concentration of the sodium nitrate in the culture broth for culturing the microorganisms having both nitrification and denitrification capabilities is in a range of 8.5-42.5 g/L, and further 20-30 g/L. In the present disclosure, a purpose of adding the sodium nitrate to the culture broth for culturing the microorganisms having both nitrification and denitrification capabilities is to enhance the ability of the bacteria to utilize nitrate ions.

As a specific embodiment of the present disclosure, a culture broth for culturing the ureolytic bacteria includes a nutrient broth powder, sodium chloride, urea and distilled water; a mass concentration of the nutrient broth powder in the culture broth for culturing the ureolytic bacteria is in a range of 1-10 g/L, and further 5-8 g/L; a mass concentration of the sodium chloride in the culture broth for culturing the ureolytic bacteria is in a range of 5-20 g/L, and further 10-15 g/L; and a mass concentration of the urea in the culture broth for culturing the ureolytic bacteria is in a range of 6-31 g/L, and further 10-25 g/L. As a specific embodiment of the present disclosure, culturing the microorganisms having both nitrification and denitrification capabilities and the ureolytic bacteria is conducted at a temperature of 20-40° C., and further 30-35° C. As a specific embodiment of the present disclosure, the culture of the microorganisms having both nitrification and denitrification capabilities has a concentration of 10⁷-10⁸ CFU/L, and the culture of the ureolytic bacteria has a concentration of 10⁷-10⁸ CFU/L.

In the present disclosure, there is no special requirement for a process of the first mixing, as long as uniform mixing can be achieved.

In the present disclosure, after the mixed culture is obtained, the mixed culture, the recycled aggregate and the mineralizing solution are subjected to a second mixing, and a resulting system is subjected to microbial mineralization to obtain the microbial mineralized modified recycled aggregate. As a specific embodiment of the present disclosure, the second mixing includes:
subjecting the mixed culture and the recycled aggregate to a third mixing, and then standing to obtain a recycled aggregate attached with the microorganisms; and
subjecting the mineralizing solution and the recycled aggregate attached with the microorganisms to a fourth mixing.

As a specific embodiment of the present disclosure, the recycled aggregate is obtained by crushing a waste concrete, and the waste concrete is a waste concrete formed from the demolition of buildings. In the present disclosure, there is no special limitation on a particle size, a crushing index, a water absorption and an apparent density of the recycled aggregate, and the recycled aggregate conventional in the art may be adopted. As a specific embodiment of the present disclosure, a mass ratio of the mixed culture to the recycled aggregate is in a range of 0.08-0.12:1, specifically 0.1:1. As a specific embodiment of the present disclosure, the third mixing is conducted by spraying the mixed culture onto a surface of the recycled aggregate or soaking the recycled aggregate in the mixed culture for 30-40 minutes.

As a specific embodiment of the present disclosure, the standing after the third mixing is conducted for 6-12 hours, and further 8-10 hours. In the present disclosure, the standing facilitates sufficient contact between the recycled aggregate and the mixed culture, enabling bacteria to fully attach to the surface of the recycled aggregate.

As a specific embodiment of the present disclosure, the mineralizing solution includes urea and calcium formate; a mass concentration of the urea in the mineralizing solution is in a range of 6-30 g/L, specifically 10 g/L, 15 g/L, 20 g/L or 25 g/L; a mass concentration of the calcium formate in the mineralizing solution is in a range of 40-45 g/L, specifically 42 g/L or 44 g/L; the mineralizing solution further includes at least one selected from the group consisting of calcium chloride, and glucose; under a condition that the calcium chloride is included in the mineralizing solution, a mass concentration of the calcium chloride in the mineralizing solution is in a range of 50-60 g/L, specifically 53 g/L, 55.5 g/L or 58 g/L; and under a condition that the glucose is included in the mineralizing solution, a mass concentration of the glucose in the mineralizing solution is in a range of 0.8-1.2 g/L, specifically 1 g/L. As a specific embodiment of the present disclosure, a ratio of a mass of the recycled aggregate to a volume of the mineralizing solution is in a range of 1 kg: 1.1-1.8 L, specifically 1 kg: 1.1 L, 1 kg: 1.2 L, 1 kg: 1.3 L, 1 kg: 1.4 L, 1 kg: 1.5 L, 1 kg: 1.6 L, 1 kg: 1.7 L or 1 kg: 1.8 L. In the present disclosure, the urea is a nutrient for the ureolytic bacteria, the calcium formate provides calcium ions and also provides a small-molecule carbon source to facilitate the generation of carbonate ions, and the glucose could improve the activity of the bacteria.

As a specific embodiment of the present disclosure, the fourth mixing is conducted by spraying the mineralizing solution onto the recycled aggregate attached with the microorganisms or soaking the recycled aggregate attached with the microorganisms in the mineralizing solution.

As a specific embodiment of the present disclosure, after the second mixing, the method further includes: mixing the resulting system after the second mixing with a pH regulator to adjust a pH value of the resulting system to 8.5-9.5, where the pH value after adjusting specifically is 9; the pH regulator includes a sodium hydroxide solution; and the sodium hydroxide solution has a molar concentration of 0.08-0.12 mol/L, specifically 0.1 mol/L. In the present disclosure, there is no special requirement for an amount of the pH regulator, as long as a desired pH value could be achieved. In the present disclosure, the pH value of the resulting system is limited to the above range to facilitate the rapid production of calcium carbonate during microbial mineralization.

As a specific embodiment of the present disclosure, the microbial mineralization is conducted by standing the resulting system after the second mixing for 7-10 days, specifically 7 days, 8 days, 9 days or 10 days. As a specific embodiment of the present disclosure, the microbial mineralization is conducted by standing the resulting system after the second mixing at a temperature of 20-40° C., specifically 20° C., 25° C., 30° C., 35° C. or 40° C.

As a specific embodiment of the present disclosure, after the microbial mineralization, the method further includes: separating an aggregate after the microbial mineralization, and then drying the aggregate to obtain the microbial mineralized modified recycled aggregate. In the present disclosure, there is no special requirement for the separating, and a process conventional in the art may be adopted. As a specific embodiment of the present disclosure, the drying is conducted at a temperature of 40-60° C., and further 45-55° C. In the present disclosure, there is no special limitation on a time for the drying, as long as a solvent on a surface of a solid can be removed.

In the present disclosure, ureolytic bacteria are selected and denitrifying bacteria with nitrification capability are screened for the microbial mineralization together, which converts ammonia gas produced by decomposition of the urea by the ureolytic bacteria into nitrate ions while ensuring the mineralization efficiency, and then secondary microbial mineralization is conducted using the nitrate ions to further strengthen the recycled aggregate without producing ammonia gas. In the present disclosure, the microbial mineralization includes the following process.

Step 1: Primary mineralization (the ureolytic bacteria decompose urea to produce calcium carbonate)

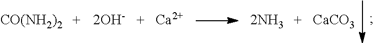

Step 2: Nitrification converts ammonia gas into nitrate ions

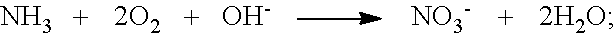

Step 3: Denitrification process realizes secondary mineralization

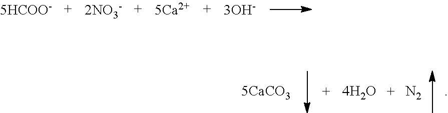

The present disclosure further provides a microbial mineralized modified recycled aggregate prepared by the method described in the above technical solutions, where the microbial mineralized modified recycled aggregate has a water absorption of 8.58-8.82%, an apparent density of 2571-2591 kg/m³ and a crushing index of 17.5-18.1%. The microbial mineralized modified recycled aggregate obtained according to the method provided by the present disclosure has a low aggregate crushing index, low water absorption and high strength; a recycled aggregate concrete formed by the microbial mineralized modified recycled aggregate has high strength and durability.

The present disclosure further provides use of the microbial mineralized modified recycled aggregate described in the above technical solutions in a recycled aggregate concrete.

To further illustrate the present disclosure, technical solutions provided by the present disclosure will be described in detail below in conjunction with examples, but these examples should not be understood as limiting the scope of the present disclosure.

### Example 1

(1) Preparation of culture: A culture broth composed of a nutrient broth powder (5 g/L), NaCl (10 g/L) and distilled water was sterilized and cooled to room temperature, and then desired bacteria were inoculated into the culture broth. When Sporosarcina pasteurii (SP) (ATCC 11859) was inoculated into the culture broth, urea was added in a concentration of 31 g/L (0.5 mol/L). A resulting mixture was incubated under shaking at 30° C. until a bacterial concentration reached 10⁷ CFU/L. When Paracoccus denitrificans (Pd) (ATCC 19367) was inoculated into the culture broth, sodium nitrate was added in a concentration of 42.5 g/L (0.5 mol/L). An obtained mixture was incubated under shaking at 30° C. until a bacterial concentration reached 10⁷ CFU/L. 20 mL of a resulting culture of Paracoccus denitrificans at the bacterial concentration of 10⁷ CFU/L was mixed with 80 mL of a resulting culture of Sporosarcina pasteurii at the bacterial concentration of 10⁷ CFU/L to obtain 100 mL of a mixed culture.

(2) 1 kg of a recycled aggregate with a crushing index of 21.2%, a water absorption of 11.9% and an apparent density of 2427.7 kg/m³ was placed in a 6 L transparent container. Then, 100 mL of the mixed culture was sprayed, and then standing was conducted for 6 hours to obtain a recycled aggregate attached with microorganisms. The recycled aggregate was obtained by crushing a waste concrete. The crushing index, the water absorption and the apparent density were tested according to GB/T25177-2010 “Recycled Coarse Aggregate for Concrete and Mortar” and GB/T14685-2022 “Pebble and Crushed Stone for Construction”.

(3) 1.3 L of a mineralizing solution (containing 20 g/L urea, 55.5 g/L calcium chloride, 42 g/L calcium formate and 1 g/L glucose) was added to a resulting mixed system obtained in step (2), and then a pH value was adjusted to 9 with a NaOH solution at a molar concentration of 0.1 mol/L. A resulting system was mixed uniformly and left to stand (for microbial mineralization) for 168 hours (7 days), and then an aggregate was taken out to obtain a microbial mineralized modified recycled aggregate.

### Example 2

A recycled aggregate was subjected to microbial mineralization according to the method of Example 1, except that a mixed culture was obtained by mixing 33.3 mL of the resulting culture of Paracoccus denitrificans at the bacterial concentration of 10⁷ CFU/L and 66.7 mL of the resulting culture of Sporosarcina pasteurii at the bacterial concentration of 10⁷ CFU/L.

### Example 3

A recycled aggregate was subjected to microbial mineralization according to the method of Example 1, except that a mixed culture was obtained by mixing 50 mL of the resulting culture of Paracoccus denitrificans at the bacterial concentration of 10⁷ CFU/L and 50 mL of the resulting culture of Sporosarcina pasteurii at the bacterial concentration of 10⁷ CFU/L.

### Example 4

A recycled aggregate was subjected to microbial mineralization according to the method of Example 1, except that Sporosarcina pasteurii (SP) (ATCC 11859) was replaced with Bacillus megaterium de Bary (Bm) (ATCC 25300); and a mixed culture was obtained by mixing 50 mL of the resulting culture of Paracoccus denitrificans at the bacterial concentration of 10⁷ CFU/L and 50 mL of a resulting culture of Bacillus megaterium de Bary at a bacterial concentration of 10⁷ CFU/L.

### Example 5

A recycled aggregate was subjected to microbial mineralization according to the method of Example 1, except that Paracoccus denitrificans (Pd) (ATCC 19367) was replaced with Pseudomonas. Sp (Psp) (ATCC 13867); and a mixed culture was obtained by mixing 50 mL of a resulting culture of Pseudomonas. Sp at a bacterial concentration of 10⁷ CFU/L and 50 mL of the resulting culture of Sporosarcina pasteurii at the bacterial concentration of 10⁷ CFU/L.

### Comparative Example 1 (Single Bacterium for Microbial Mineralization in Comparison with Example 1)

(1) Preparation of culture: A culture broth composed of a nutrient broth powder (5 g/L), NaCl (10 g/L) and distilled water was sterilized and cooled to room temperature, and then desired bacteria were inoculated into the culture broth. When Sporosarcina pasteurii (SP) (ATCC 11859) was inoculated into the culture broth, urea was added in a concentration of 31 g/L (0.5 mol/L). A resulting mixture was incubated under shaking at 30° C. until a bacterial concentration reached 10⁷ CFU/L. 100 mL of a resulting culture of Sporosarcina pasteurii at the bacterial concentration of 10⁷ CFU/L was taken.

(2) 1 kg of a recycled aggregate with a crushing index of 21.2%, a water absorption of 11.9% and an apparent density of 2427.7 kg/m³ was placed in a 20 L transparent container (due to an excessively high concentration of ammonia gas generated by this method, which would exceed a measuring range of a detector in a 6 L container, a 20 L transparent container was used instead). Then, 100 mL of the resulting culture of Sporosarcina pasteurii was sprayed, and then standing was conducted for 6 hours to obtain a recycled aggregate attached with microorganisms. The recycled aggregate was obtained by crushing a waste concrete.

(3) 1.3 L of a mineralizing solution (consisting of 110 g/L calcium chloride and 1 g/L glucose) was added to a resulting mixed system obtained in step (2), and then a pH value was adjusted to 9 with a NaOH solution at a molar concentration of 0.1 mol/L. A resulting system was mixed uniformly and left to stand (for microbial mineralization) for 168 hours (7 days), and then an aggregate was taken out to obtain a microbial mineralized modified recycled aggregate.

### Comparative Example 2

A recycled aggregate was subjected to microbial mineralization according to the method of Example 1, except that a mixed culture was obtained by mixing 10 mL of the resulting culture of Paracoccus denitrificans at the bacterial concentration of 10⁷ CFU/L and 90 mL of the resulting culture of Sporosarcina pasteurii at the bacterial concentration of 10⁷ CFU/L. Due to an excessively high concentration of ammonia gas generated by this method, which would exceed a measuring range of a detector in a 6 L container, a volume of the transparent container was increased from 6 L to 20 L.

### Comparative Example 3

A recycled aggregate was subjected to a microbial mineralization treatment according to the method of Example 1, except that a mixed culture was obtained by mixing 90 mL of the resulting culture of Paracoccus denitrificans at the bacterial concentration of 10⁷ CFU/L and 10 mL of the resulting culture of Sporosarcina pasteurii at the bacterial concentration of 10⁷ CFU/L. Due to an excessively high concentration of ammonia gas generated by this method, which would exceed a measuring range of a detector in a 6 L container, a volume of the transparent container was increased from 6 L to 20 L.

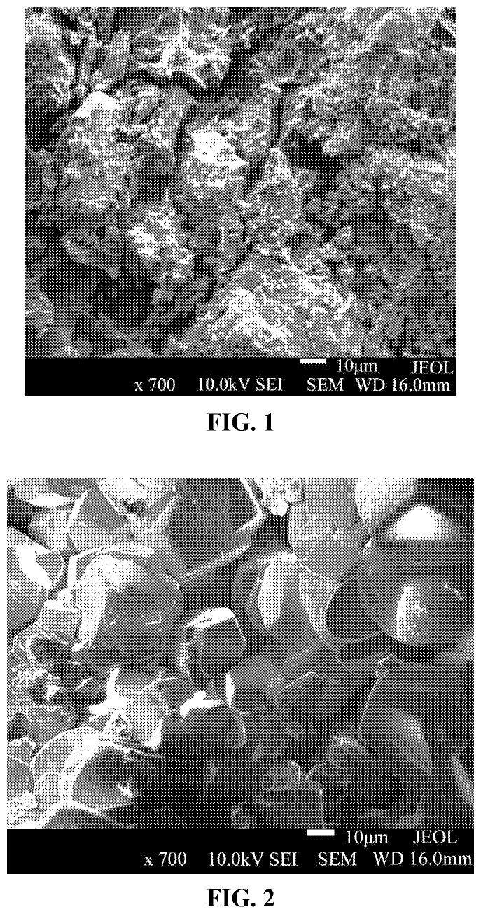

A surface of the recycled aggregate in Example 1 was examined by scanning electron microscopy (SEM), and an SEM image was obtained, as shown in FIG. 1. A surface of the microbial mineralized modified recycled aggregate prepared in Example 3 was examined by scanning electron microscopy (SEM), and an SEM image was obtained, as shown in FIG. 2. As can be seen from FIG. 1 and FIG. 2, composite bacteria fill a loose structure of the recycled aggregate by microbial mineralization, forming a dense calcium carbonate layer.

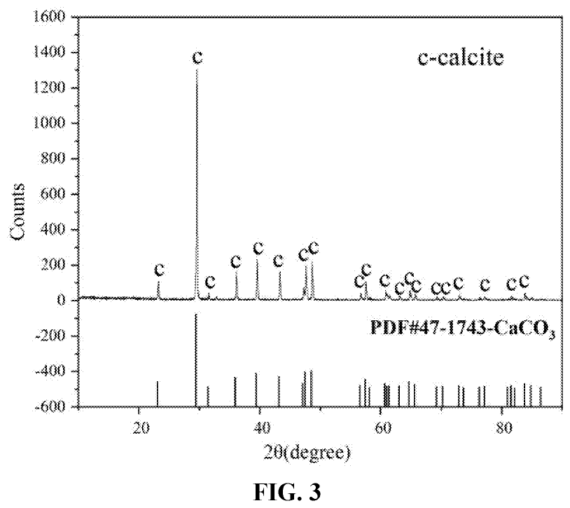

A calcium carbonate protective layer on the surface of the microbial mineralized modified recycled aggregate prepared in Example 3 was examined by X-ray diffraction, and an XRD pattern was obtained, as shown in FIG. 3.

As can be seen from FIG. 3 in combination with FIG. 2, the microbial mineralization was conducted according to the method provided by the present disclosure, an ingredient produced by a composite bacterial strain on the surface of the recycled aggregate, which fills pores and cracks on the surface of the recycled aggregate, is calcium carbonate. Combined with XRD and SEM observation results, calcium carbonate crystals exist in a form of calcite.

During the microbial mineralization in Comparative Examples 1-3, concentrations of ammonia gas in the 20 L container were detected at 2 hours, 4 hours, 12 hours, 24 hours, 36 hours, 48 hours, 60 hours, 72 hours, 96 hours and 168 hours using a Smart Sensor ammonia gas detector ST9500, respectively, and detected results were converted to concentrations of the ammonia gas in a 6 L container. Results are listed in Table 1.

During the microbial mineralization in Examples 1-5, concentrations of ammonia gas in the 6 L container were detected at 2 hours, 4 hours, 12 hours, 24 hours, 36 hours, 48 hours, 60 hours, 72 hours, 96 hours and 168 hours using the Smart Sensor ammonia gas detector ST9500, respectively. Results are listed in Table 1.

An apparent density, a water absorption and a crushing index of the microbial mineralized modified recycled aggregate obtained from Examples 1-5 and Comparative Examples 1-3 were tested according to GB/T25177-2010 “Recycled Coarse Aggregate for Concrete and Mortar” and GB/T14684-2022 “Sand for Construction”. Results are listed in Table 2.

TABLE 1
Concentration of ammonia gas in 6 L container during microbial
mineralization in Examples 1-5 and Comparative Examples 1-3

| | Concentration of ammonia gas in 6 L container at different test times (ppm) | | | | | | | | | | | |
|---|---|---|---|---|---|---|---|---|---|---|---|---|
| | 2 | 4 | 12 | 24 | 36 | 48 | 60 | 72 | 96 | 120 | 144 | 168 |
| Example | hours | hours | hours | hours | hours | hours | hours | hours | hours | hours | hours | hours |
| Example 1 (4 | 18.4 | 35.2 | 62.2 | 58.8 | 36.7 | 34.8 | 24.7 | 21.9 | 19.1 | 16.3 | 13.5 | 13.7 |
| SP:1 Pd) | | | | | | | | | | | | |
| Example 2 (2 | 16.5 | 28.2 | 44.8 | 48.5 | 36 | 35.8 | 31.7 | 26.4 | 21.1 | 11.8 | 10.5 | 11.2 |
| SP:1 Pd) | | | | | | | | | | | | |
| Example 3 (1 | 17.3 | 21.4 | 20.4 | 16.6 | 15.8 | 12.9 | 11.8 | 13.1 | 14.4 | 10.7 | 11.0 | 10.2 |
| SP:1 Pd) | | | | | | | | | | | | |
| Example 4 (1 | 16.4 | 19.8 | 27.2 | 22.7 | 23.1 | 12.9 | 12.2 | 13.5 | 13.9 | 9.2 | 8.6 | 8.9 |
| Bm:1 Pd) | | | | | | | | | | | | |
| Example 5 (1 | 18.2 | 19.8 | 23.4 | 18.1 | 15.7 | 15.2 | 14.3 | 15.1 | 14.8 | 8.8 | 7.7 | 7.7 |
| SP:1 Psp) | | | | | | | | | | | | |
| Comparative | 279.2 | 251.6 | 307.2 | 272 | 197.2 | 180.8 | 84.4 | 46.9 | 48.5 | 49.1 | 47.9 | 48.8 |
| Example 1 | | | | | | | | | | | | |
| Comparative | 256.3 | 230.7 | 271.2 | 267.6 | 193.1 | 136.3 | 79.5 | 22.7 | 22.7 | 22.7 | 22.7 | 22.7 |
| Example 2 | | | | | | | | | | | | |
| Comparative | 15.6 | 13.1 | 12.6 | 8.9 | 2.8 | 2.7 | 2.5 | 2.3 | 2.1 | 2.9 | 2.7 | 2.5 |
| Example 3 | | | | | | | | | | | | |

TABLE 2
Performance parameters of microbial mineralized modified recycled
aggregate of Examples 1-5 and Comparative Examples 1-3

| | | | | | | | Maximum |
|---|---|---|---|---|---|---|---|
| | | | | | | | ammonia gas |
| | Water absorption | | Apparent density | | Crushing index % | | concentration |
| | Value | Decrease | Value | Increase | | Decrease | in 6 L container |
| Example | % | ratio | kg/m³ | ratio | Value | ratio | ppm |
| Example 1 (4 SP:1 Pd) | 8.58 | 27.9% | 2590.8 | 6.7% | 17.7 | 16.5% | 62.2 |
| Example 2 (2 SP:1 Pd) | 8.70 | 26.9% | 2579.4 | 6.2% | 17.9 | 15.6% | 48.4 |
| Example 3 (1 SP:1 Pd) | 8.71 | 26.8% | 2587.4 | 6.6% | 17.5 | 17.5% | 20.4 |
| Example 4 (1 Bm:1 Pd) | 8.78 | 26.2% | 2588.6 | 6.6% | 17.8 | 16.0% | 27.2 |
| Example 5 (1 SP:1 Psp) | 8.82 | 25.9% | 2571.7 | 5.9% | 18.1 | 14.6% | 23.4 |
| Comparative Example 1 | 8.59 | 27.8% | 2590.8 | 6.7% | 17.7 | 16.5% | 307.2 |
| Comparative Example 2 | 8.61 | 27.6% | 2589.4 | 6.7% | 17.8 | 16.0% | 271.2 |
| Comparative Example 3 | 10.64 | 10.6% | 2511.4 | 3.4% | 20.9 | 1.4% | 15.6 |

As can be seen from Table 1 in combination with Table 2, ureolytic bacteria could effectively improve the performance of a recycled aggregate, but a large amount of ammonia gas is generated during microbial mineralization. When an amount of microorganisms having both nitrification and denitrification capabilities is too small, their ability to decompose the ammonia gas is insufficient. When the amount of the microorganisms having both nitrification and denitrification capabilities is too large, their ability to modify the recycled aggregate is insufficient. This is because when an amount of the ureolytic bacteria is too small, a primary microbial mineralization proceeds slowly, which in turn results in insufficient production of nitrate ions. In the absence of additional nitrate ion supplementation, a secondary microbial mineralization also proceeds slowly.

When a volume ratio at equal concentrations of the culture of the microorganisms having both nitrification and denitrification capabilities to the culture of the ureolytic bacteria in a mixed culture is limited to 2:1 to 1:4 according to the method provided by the present disclosure, the performance of the recycled aggregate is effectively improved, and an amount of ammonia gas during the microbial mineralization is effectively controlled simultaneously.

Using the recycled aggregate from Example 3 as a raw material, a mixture was prepared according to a mass ratio of a cement, water, a fine aggregate and a recycled coarse aggregate being 400:170:720:1095, and then the mixture was cast into cubic test blocks with a side length of 150 mm. After curing for 28 days, a compressive strength test was conducted according to “Standard for Test Methods of Concrete Physical and Mechanical Properties” (GB/T 50081-2019). A water absorption test was conducted according to “Recycled Coarse Aggregate for Concrete” (GB/T 25177-2010). Test results showed that an average 28-day compressive strength was 29.2 MPa, and a water absorption was 8.9%.

Using the microbial mineralized modified recycled aggregate treated in Example 3 as a raw material, a mixture was prepared according to a mass ratio of a cement, water, a fine aggregate and a recycled coarse aggregate being 400:170:720:1167, and then the mixture was cast into cubic test blocks with a side length of 150 mm. After curing for 28 days, a compressive strength test was conducted according to “Standard for Test Methods of Concrete Physical and Mechanical Properties” (GB/T 50081-2019). A water absorption test was conducted according to “Recycled Coarse Aggregate for Concrete” (GB/T 25177-2010). Test results showed that an average 28-day compressive strength was 33.2 MPa, and a water absorption was 7.2%.

As can be seen from test block results, test blocks of a recycled concrete prepared by using a microbial mineralized modified recycled aggregate (obtained by treating the recycled aggregate by the method of the present disclosure) as a raw material exhibit a high compressive strength and a low water absorption.

Although the present disclosure is described in detail in conjunction with the foregoing embodiments, they are only a part of, not all of, the embodiments of the present disclosure. Other embodiments could be obtained based on these embodiments without inventive efforts, and all of these embodiments shall fall within the scope of the present disclosure.

What is claimed is:

1. A method for preparing a microbial mineralized modified recycled aggregate, comprising the following steps:
subjecting a culture of microorganisms having both nitrification and denitrification capabilities and a culture of ureolytic bacteria to a first mixing to obtain a mixed culture, wherein a volume ratio at equal concentrations of the culture of the microorganisms having both nitrification and denitrification capabilities to the culture of the ureolytic bacteria in the mixed culture is in a range of 2:1 to 1:4; and
subjecting the mixed culture, a recycled aggregate and a mineralizing solution to a second mixing, and subjecting a resulting system to microbial mineralization to obtain the microbial mineralized modified recycled aggregate, wherein the mineralizing solution comprises urea and calcium formate.

2. The method for preparing the microbial mineralized modified recycled aggregate of claim 1, wherein the microorganisms having both nitrification and denitrification capabilities comprise at least one selected from the group consisting of Paracoccus denitrificans, and Pseudomonas. Sp; and
the ureolytic bacteria comprise at least one selected from the group consisting of Sporosarcina pasteurii, and Bacillus megaterium de Bary.

3. The method for preparing the microbial mineralized modified recycled aggregate of claim 1, wherein the culture of the microorganisms having both nitrification and denitrification capabilities has a concentration of 10⁷-10⁸ CFU/L (colony-forming unit per liter); and
the culture of the ureolytic bacteria has a concentration of 10⁷-10⁸ CFU/L.

4. The method for preparing the microbial mineralized modified recycled aggregate of claim 2, wherein the culture of the microorganisms having both nitrification and denitrification capabilities has a concentration of 10⁷-10⁸ CFU/L; and
the culture of the ureolytic bacteria has a concentration of 10⁷-10⁸ CFU/L.

5. The method for preparing the microbial mineralized modified recycled aggregate of claim 1, wherein the second mixing comprises:
subjecting the mixed culture and the recycled aggregate to a third mixing, and then standing to obtain a recycled aggregate attached with the microorganisms; and
subjecting the mineralizing solution and the recycled aggregate attached with the microorganisms to a fourth mixing.

6. The method for preparing the microbial mineralized modified recycled aggregate of claim 1, wherein a mass concentration of the urea in the mineralizing solution is in a range of 6-30 g/L, and a mass concentration of the calcium formate in the mineralizing solution is in a range of 40-45 g/L; and a ratio of a mass of the recycled aggregate to a volume of the mineralizing solution is in a range of 1 kg: 1.1-1.8 L; and
a mass ratio of the mixed culture to the recycled aggregate is in a range of 0.08-0.12:1.

7. The method for preparing the microbial mineralized modified recycled aggregate of claim 5, wherein a mass concentration of the urea in the mineralizing solution is in a range of 6-30 g/L, and a mass concentration of the calcium formate in the mineralizing solution is in a range of 40-45 g/L; and a ratio of a mass of the recycled aggregate to a volume of the mineralizing solution is in a range of 1 kg: 1.1-1.8 L; and
a mass ratio of the mixed culture to the recycled aggregate is in a range of 0.08-0.12:1.

8. The method for preparing the microbial mineralized modified recycled aggregate of claim 6, wherein the mineralizing solution further comprises at least one selected from the group consisting of calcium chloride, and glucose;
under a condition that the calcium chloride is comprised in the mineralizing solution, a mass concentration of the calcium chloride in the mineralizing solution is in a range of 50-60 g/L; and
under a condition that the glucose is comprised in the mineralizing solution, a mass concentration of the glucose in the mineralizing solution is in a range of 0.8-1.2 g/L.

9. The method for preparing the microbial mineralized modified recycled aggregate of claim 1, wherein after the second mixing, the method further comprises: mixing the resulting system after the second mixing with a pH regulator to adjust a pH value of the resulting system to 8.5-9.5, wherein the pH regulator comprises a sodium hydroxide solution.

10. The method for preparing the microbial mineralized modified recycled aggregate of claim 1, wherein the microbial mineralization is conducted by standing the resulting system after the second mixing for 7-10 days at a temperature of 20-40° C.

11. A microbial mineralized modified recycled aggregate prepared by the method of claim 1, wherein the microbial mineralized modified recycled aggregate has a water absorption of 8.58-8.82%, an apparent density of 2571-2591 kg/m³ and a crushing index of 17.5-18.1%.

12. The microbial mineralized modified recycled aggregate of claim 11, wherein the microorganisms having both nitrification and denitrification capabilities comprise at least one selected from the group consisting of Paracoccus denitrificans, and Pseudomonas. Sp; and
the ureolytic bacteria comprise at least one selected from the group consisting of Sporosarcina pasteurii, and Bacillus megaterium de Bary.

13. The microbial mineralized modified recycled aggregate of claim 11, wherein the culture of the microorganisms having both nitrification and denitrification capabilities has a concentration of 10⁷-10⁸ CFU/L; and
the culture of the ureolytic bacteria has a concentration of 10⁷-10⁸ CFU/L.

14. The microbial mineralized modified recycled aggregate of claim 11, wherein the second mixing comprises:
subjecting the mixed culture and the recycled aggregate to a third mixing, and then standing to obtain a recycled aggregate attached with the microorganisms; and
subjecting the mineralizing solution and the recycled aggregate attached with the microorganisms to a fourth mixing.

15. The microbial mineralized modified recycled aggregate of claim 11, wherein a mass concentration of the urea in the mineralizing solution is in a range of 6-30 g/L, and a mass concentration of the calcium formate in the mineralizing solution is in a range of 40-45 g/L; and a ratio of a mass of the recycled aggregate to a volume of the mineralizing solution is in a range of 1 kg: 1.1-1.8 L; and
a mass ratio of the mixed culture to the recycled aggregate is in a range of 0.08-0.12:1.

16. The microbial mineralized modified recycled aggregate of claim 15, wherein the mineralizing solution further comprises at least one selected from the group consisting of calcium chloride, and glucose;
under a condition that the calcium chloride is comprised in the mineralizing solution, a mass concentration of the calcium chloride in the mineralizing solution is in a range of 50-60 g/L; and
under a condition that the glucose is comprised in the mineralizing solution, a mass concentration of the glucose in the mineralizing solution is in a range of 0.8-1.2 g/L.

17. The microbial mineralized modified recycled aggregate of claim 11, wherein after the second mixing, the method further comprises: mixing the resulting system after the second mixing with a pH regulator to adjust a pH value of the resulting system to 8.5-9.5, wherein the pH regulator comprises a sodium hydroxide solution.

18. The microbial mineralized modified recycled aggregate of claim 11, wherein the microbial mineralization is conducted by standing the resulting system after the second mixing for 7-10 days at a temperature of 20-40° C.
